(19) United States
(12) Patent Application Publication
(10) Pub. No.: US 2017/0295822 A1
(43) Pub. Date: Oct. 19, 2017

(54) NATURAL, BIODEGRADABLE BEADLETS AND PROCESS TO MANUFACTURE

(71) Applicant: Corn Products Development, Inc., Jabaquara (BR)

(72) Inventors: Sarfaraz Patel, Hillsborough, NJ (US); John Pantina, Somerset, NJ (US)

(73) Assignee: Corn Products Development, Inc., Jabaquara (BR)

(21) Appl. No.: 15/130,071

(22) Filed: Apr. 15, 2016

(51) Int. Cl. A23J 3/14; C11D 3/00; A61Q 19/10; A61K 8/64; A23J 1/12; A61Q 5/02; A61Q 5/12; A61K 9/14; C11D 3/382; A61Q 11/00

(52) U.S. Cl. CPC A23J 3/14; C11D 3/382; C11D 3/001; A61Q 19/10; A61Q 11/00; A23J 1/12; A61Q 5/02; A61Q 5/12; A61K 9/146; A61K 8/645; A61K 2800/10

(57) ABSTRACT

The present invention uses natural materials, namely corn protein concentrates, as bead materials, providing a greener solution to synthetic beads. The present invention further eliminates the need for solvents and other potentially environmentally dangerous chemicals by producing the beads directly from a corn protein concentrate cake.

### BACKGROUND OF THE INVENTION

The present invention relates to spherical beads produced from corn protein concentrate which have a substantially uniform diameter and may be used in a broad range of end use applications. Such beads are produced directly from the corn protein concentrate cake without the need to use solvents or other chemicals.

Spherical beads having diameters of 10-5000 microns are typically produced using synthetic materials, which are not biodegradable. These beads are often washed down the drain into the sewage systems, and are not easily recovered during waste water treatment. The beads collect in lakes, rivers and oceans, where they are dangerous to marine life.

This growing environmental problem of marine ecosystem contamination has justified the need for natural alternatives. Recently, there has been a widespread movement to ban use of synthetic beads, particularly in personal care applications.

Production of synthetic beads typically uses solvents and other chemicals which are not environmentally friendly. The process relies upon the phenomenon of fluid flow instabilities to promote lump formation. The lumps then form into spherical beads due to surface energy. The solvents and other chemicals must then be disposed of without contamination of our water systems.

There is thus a need for a bio-friendly bead, which is not only made from natural materials, but is preferably biodegradable. There is also a need for a more environmentally friendly process of making beads, without the use of significant solvents or chemicals.

### SUMMARY OF THE INVENTION

The present invention uses natural materials, namely corn protein concentrates, as bead materials, providing a greener solution to synthetic beads. The present invention further eliminates the need for solvents and other potentially environmentally dangerous chemicals by producing the beads directly from a corn protein concentrate cake.

Corn protein concentrate, as used herein, is intended to mean a material derived from corn with a protein content greater or equal to 75% and starch content less than 1%, each by weight, dry solids basis (dsb). Protein is measured using Leco TruMac® N series equipment.

Water-rich, as used herein, is intended to mean a phase which contains at least 60% (w/w) water prior to dispersion of the beads.

Hydrophobic, as used herein, is intended to mean material with high affinity for oil, fat and other non-polar materials. Hydrophobicity of corn protein is usually defined by how much hydrophobic amino acid (such as alanine, isoleucine, leucine, valine, phenylalanine, tryptophan and tyrosine) is present in the protein.

Native, as used herein, is intended to mean unmodified protein as extracted from the corn.

Personal care, as used herein, is intended to mean a product intended for application to a human body, useful for personal hygiene and/or beautification.

### BRIEF DESCRIPTION OF THE DRAWINGS

FIG. 1 depicts a flow diagram of one method of making corn protein concentrate beads.

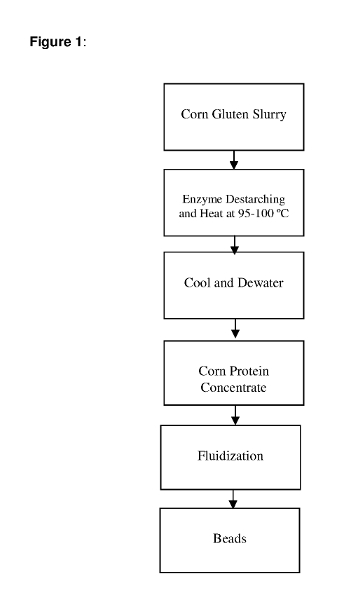

FIG. 2 depicts particle size distribution of beads produced in Examples 1 and 2.

### DETAILED DESCRIPTION OF THE INVENTION

The present invention uses natural materials, namely corn protein concentrate, as bead material, providing a greener solution to synthetic beads. The present invention further eliminates the need for solvents and other potentially environmentally dangerous chemicals by producing the beads directly from a corn protein concentrate cake.

The base material used for the present invention is any corn protein concentrate. The corn protein concentrate may be derived from any corn, including dent, waxy or high amylose. As used herein, waxy corn is that in which the starch contains less than 5% (w/w), particularly less than 2% (w/w), and more particularly less than 1% (w/w) amylose. As used herein, high amylose corn is that in which the starch contains at least 50% (w/w), particularly at least 70% (w/w), more particularly at least 80% (w/w), and most particularly at least 90% (w/w) amylose.

The byproducts of corn processing, such as dry milling, wet milling, alkaline processing and dry grind processing include corn proteins. These protein byproducts include corn gluten meal, corn gluten feed, and distillers dried grains. These byproducts contain different amounts of protein. Corn proteins may then be concentrated by any process known in the art, including without limitation by those disclosed in U.S. Pat. Nos. 3,535,305 and 3,782,964, both incorporated by reference herein. U.S. Pat. No. 3,535,305 discloses a process comprising mixing the gluten with an aqueous solvent, heating the gluten/solvent mixture at a temperature of 50-75° C., mechanically separating the undissolved solids, chilling the resulting crude zein solution, and removing the supernatant liquid from the zein solvent layer. U.S. Pat. No. 3,782,964 discloses a process comprising subjecting the gluten to the hydrolytic action of an enzyme to hydrolyze the starch; and heating the enzyme-treated gluten to a temperature of at least 95° C. Another possible process is by extraction with an aqueous organic solvent such as ethyl or isopropyl alcohol, with or without subsequent re-extraction with a fat solvent such as hexane or benzene, recovery of residual volatile hydrocarbons from the extract by distillation and precipitation of the proteins by mixing the remaining alcoholic solution with water. According to solubility in different solvents, four major classes of protein namely prolamine, albumin, globulin and glutelin are found in maize. In one embodiment, the corn protein concentrate used in the process concentrates all four types of proteins.

The corn protein from such gluten slurry is concentrated by at least partially destarching by use of an enzyme such as an amylase, a diastase, or a malt. The enzyme hydrolyzes the starch into smaller polymers or sugars so that it may be washed away. In one embodiment, an amylase is used. In another embodiment, the amylase is a glucoamylase or an alpha-amylase and in yet another embodiment, an alpha amylase is used. In still yet another embodiment, a thermally resistant alpha amylase is used, such as Termamyl 120L. When selecting an enzyme, it is desirable to use one which is relatively low in proteolytic activity to minimize protein loss. The conditions of such destarching, such as percent solids, temperature and pH, are dependent upon the type of enzyme used, the amount of destarching desired, and the quantity of starch present. In one embodiment, the resulting corn protein concentrate slurry contains at least 65%, in another embodiment at least about 70%, and in yet another embodiment at least about 75% (w/w dsb) protein. In one embodiment, the resulting corn protein concentrate slurry contains less than 5%, in another embodiment less than 3%, and in yet another embodiment less than 2% (w/w dsb) starch. The remainder of the corn protein concentrate comprises fiber, fat, ash and water (moisture).

The corn protein concentrate slurry may optionally be treated to at least partially remove undesirable flavors and odors (purification) using processes known in the art. In one embodiment, the corn protein concentrate slurry is heated to at least 95° C. to at least partially remove the undesirable “gluten” odor and/or flavor. In one such embodiment, the corn protein concentrate slurry is heated by boiling; and in another by exposure to steam. Optionally, the corn protein concentrate slurry may be washed, either before or after any purification step. Purification may also be conducted at any other stage on the protein, including in the bead stage, as long as the purification method does not adversely affect the resultant beads.

The corn protein concentrate slurry is next dewatered to yield a corn protein concentrate cake. Dewatering may be done using any process known in the art, such as filtering and/or drying. In one useful embodiment, the slurry is dewatered using positive air pressure, such as with a Pneumapress. In another useful embodiment, the slurry is dewatered by vacuum and/or air drying.

Due to the temperature sensitivity of corn proteins, dewatering should be done under conditions which will not denature the protein. As the percent moisture is decreased, the corn protein is more sensitive to temperatures. In one embodiment, dewatering is conducted at a temperature of less than 125° C., and in another embodiment at a temperature of less than 100° C.

Dewatering is most effective near the isoelectric point of the protein. The isoelectric point of corn protein is typically in the pH range of about 5.2 to about 5.5. Dewatering is continued to the desired end point which, in one embodiment is from about 30% to about 60% dry solids, in another embodiment from about 40% to about 60% dry solids, and in another embodiment is from about 45% to about 55% dry solids. Dewatering is not necessary if the moisture content of the corn protein is already low, e.g., from about 30% to about 60% dry solids.

The moist cake is then fluidized, either with gas or mechanically. In one embodiment, the fluidization is by mechanical means. If gas fluidization is use, the fluidizing gas may be any gas used in the industry to fluidize starch; in one embodiment is selected from the group consisting of air, or low oxygen air (particularly below the Limiting Oxygen Concentration of 12% (v/v), and inert gases. Such fluidization allows the beads to form directly from the cake without the use of solvent or other chemicals. The fluidization also further dries the beads, efficiently and uniformly, to result in beads with the desired moisture content. In one embodiment, the resultant beads have a moisture content of 5-30% (dsb). In another embodiment, the resultant beads have a moisture content of 20-30% (dsb). The beads may be fluidized using any equipment known in the art to fluidize a powder, and which will result in beads with the desired characteristics. Useful equipment includes, without limitation, a Littleford reactor and Procesall blending and drying equipment. The conditions should be chosen so as to not degrade or other negatively impact the resultant beads. Typical temperature are in the range of 60-75° C. and typical time is in the range of 2-4 hours, depending upon a number of factors including the desired moisture of the final bead product. Typically the fluidization is conducted at or below ambient pressure (1 atm) and in one embodiment is in the range of 0.001-0.76 m Hg in vacuum.

In order to take into account differences between different reactor volumes and agitation speeds, Froude number is calculated. Froude number is a dimensionless number calculated using agitation speed and reactor volume as main parameters. It is defined as ratio of flow inertia to external field. Froude number is defined by following formula:

Froude Number=(r*ω²)/g (1)

where r is radius of reactor impeller, ω is impeller frequency in RPM and g is acceleration due to gravity. Typically a lower Froude number means lower degree of fluidization resulting in higher average diameter and higher Froude number leads to lower average diameter values. Suitable Froude number values for the present invention are in the range of 2-40 for beadlet formation.

An optional step is to use a solvent, such as hexane or acetone, to remove the yellow color associated with many corn proteins. Such decoloring may be conducted on the corn protein (concentrate) at any time during the process or on the resultant beads. Other purification steps known in the art may also be used on the resultant beads. In one embodiment, the beads are washed prior to putting into an end use application. In one embodiment, the beads are produced without use of chemicals other than for pH adjustment. The resultant beads exhibit excellent physicochemical properties including uniform color and size distribution. The conditions of the manufacture may be controlled to provide the desired bead size. In one embodiment, the mean bead size is 600-700 microns (μm); in other embodiments, 700-800 μm, 800-900 μm, 900-1000 μm, 1000-1100 μm, 500-600 μm, 400-500 μm, or 300-400 μm. In one embodiment, the size distribution is ±100 μm; in other embodiments ±200 μm, ±300 μm, +400 μm, or ±500 μm. The beads may be sieved to control particle size and/or distribution. Mean particle size and distribution of the beads is measured by the method set forth in the examples section.

The Froude number is varied between 2 and 40 to obtain beads with different diameters. In one embodiment, a Froude number of 13 resulted in an average bead diameter of 600 microns. The resultant beads are natural. The resultant beads are biodegradable. The resultant beads are substantially spherical. The beads are hydrophobic. In one embodiment, the resultant beads are porous.

The circularity or roundness of a shape is measured by its difference from a perfect sphere. Using ImageJ software, commercially available from National Institutes of Health (http://imagej.nih.gov/ij/) and microscopic images of beads, area and perimeter values of beadlets are determined and circularity is calculated according to equation 2.

$$Circularity = 4 π [ \frac{A}{{p}^{2}} ] \qquad ( 2 )$$

where A is area and P is perimeter of bead. A perfect circle has circularity value of 1. The circularity of beadlets is calculated to be between 0.75-0.90.

The beads are water insoluble at ambient temperature (25° C.), yet dispersible. This allows the beads to be dispersed throughout a water or water rich medium, such as a mixture of water and alcohol, to form a substantially homogeneous yet substantially non-aggregated suspension of beads.

The beads may be added for visual effects/appearance, for exfoliating and scrubbing purposes, and to deliver oils and oil soluble materials in a variety of end use applications, including without limitation, personal care applications, foods and beverages, pharmaceuticals, and home care. The beads may be used as substitutes for synthetic beads. In one embodiment, the beads are used in personal care applications such as shampoos, conditioners, body washes, body scrubs including facial scrubs, cosmetics, oral care including toothpastes, soaps, and moisturizers. In another embodiment, the beads are used in food applications, such as in beverages and gels. In yet another embodiment, the beads are used in home care applications such as detergents, fabric softeners and cleaning products.

Porous beads may also be used for other purposes known in the art, such as a plating agent to absorb oils, as an anticaking or flow agents of powders, and as a process aid to prevent sticking or clumping to equipment or during processing, shipping and storage. When porous beads are produced, the beads may also be used to deliver oils and oil soluble materials as well as water, aqueous solutions, and water soluble materials, including without limitation flavors, fragrances, colors, and vitamins.

The beads may be used in any amount desired, but typically will be used as a minor component of a product. In one embodiment, the beads are used at less than 25% (w/w), in another embodiment less than 10% (w/w), of the product. In one embodiment, the beads are used in an amount of at least 0.1% (w/w), in another embodiment at least 1% (w/w), of the product. The other component(s) of the product are those conventionally used and in one embodiment includes water. In a food or beverage, the product contains at least one other edible component.

### EXAMPLES

The following examples are presented to further illustrate and explain the present invention and should not be taken as limiting in any regard. All percents used are on a weight/weight basis.

The following tests were used throughout the examples:

### Mean Particle Size and Distribution:

Mean particle size distribution is calculated using Malvern Mastersizer2000 (Malvern Instruments, UK) which measures particle size and shape using laser light differaction. During the laser diffraction measurement, particles are passed through a focused laser beam. These particles scatter light at an angle that is inversely proportional to their size. The principle of laser differaction and other relevant details are well documented in literature.

### Example 1

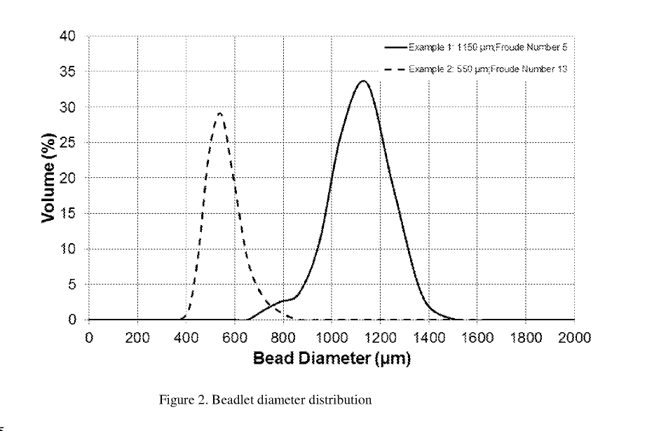

Crude gluten cake was obtained by wet milling of non genetically modified waxy corn. The wet milling procedure is well described in literature. The protein content of material was found to be 62%. Total protein content was determined using nitrogen analysis on LECO TruMac® N series equipment. The cake was slurried in water at 14-15% solids and pH adjusted to 6 using sodium hydroxide. Bacterial alpha amylase, Termamyl 120L (by Novozyme) was added to slurry in a quantity of 0.05% based on total dry solids. The mixture was kept at 80° C. for enzyme to react with slurry for one hour. The temperature of slurry was raised to about 100° C. by steam injection for a period of one hour. The resulting mixture was filtered hot through a flate and prame filter press and protein was washed with room temperature water. A small quantity of this material was dried and protein content was determined to be 75% on dry basis using Leco TruMac® N series equipment. This wet cake with moisture 55-58% was then fed to Littleford reactor. The agitation speed was maintained to Froude number 5. The protein material was then heated to 60° C. for two hours under vacuum (0.736 m Hg). The reaction was stopped after two hours. Moisture of final product was 15-18%. Average particle size of product was 1150 micron with diameter ranging from 700-1400 microns. The particles size distribution is shown in FIG. 2.

### Example 2

The gluten slurry from example 1 is treated with amyloglucosidase enzyme AMG 300 (by Novozyme) at 60° C. in addition to Termamyl 120L at 80° C. and corn protein material with 80% protein content (dry basis) was obtained. Wet cake with moisture 55-58% was then fed to Littleford reactor. The agitation speed was maintained to Froude number 13. The protein material was then heated to 60° C. for three hours under vacuum (0.736 m Hg). Particle size distribution of resulting product was measured. Moisture of final product was 12-15%. Particle diameter was measured between 400-1400 microns with average particle diameter 550 microns. The particles size distribution is shown in FIG. 2.

1.-20. (canceled)

21. A process comprising:
a. extracting protein from corn;
b. concentrating the protein to a corn protein concentrate comprising at least 65 (w/w dsb) protein;
c. adjusting the moisture content of the corn protein concentrate to yield a corn protein concentrate cake with a moisture content of 30% to about 60% by weight dry solids; and
d. fluidizing the cake to form beads.

22. The process of claim 21, wherein the corn protein concentrate is destarched to less than 5% starch (w/w dsb).

23. The process of claim 21, wherein the corn protein concentrate is purified to remove flavor and/or odor.

24. The process of claim 21, wherein the moisture is adjusted by dewatering at a temperature of less than 125° C.

25. The process of any claim 21, wherein the moisture is adjusted by dewatering the dewatering conducted at the isoelectric point of the corn protein.

26. The process of claim 25, wherein the dewatering is conducted at a pH of about 5.2 to about 5.5.

27. The process of claim 21, wherein the fluidization is by gas.

28. The process of claim 21, wherein the fluidization is by mechanical means.

29. The process of claim 21, wherein the fluidization allows the beads to form directly from the cake without the use of a solvent or another chemical.

30. The process of claim 21, wherein the fluidization in conducted at a temperature of about 60-75° C. for a period of about 2-4 hours.

31. The process of claim 21, wherein the fluidization in conducted at or below ambient pressure.

32. The process of claim 21, wherein the fluidization in conducted at a Froude number of about 2-40.

33. The process of claim 21, wherein the resultant heads have a moisture content of 5-30% (by weight dsb).

34. The process of claim 21, wherein the resultant beads have a particle size of 300-1100 microns.

35. The process of claim 21, wherein the resultant beads have a particle size distribution of about ±100 to ±500 microns.

36. The process claim 21, wherein the resultant beads have a circularity of about 0.75-0.90.

37. A composition comprising the beads produced by the process of any one of the preceding claims and a conventionally used ingredient, wherein the beads are present in an amount of less than 25% (w/w) of the composition.

38. The composition of claim 37, wherein the composition is selected from the group consisting of personal care products, foods, beverages, pharmaceuticals and home care products.

39. The composition of claim 37, wherein the composition is selected from the group consisting of a plating agent to absorb oils, an anticaking or flow agent, a process aid to prevent sticking or clumping, a delivery agent for oils or oil-soluble materials, and a delivery agent for water, aqueous solutions, or water soluble materials.

40. The composition of claim 37, wherein the composition delivers a material selected from the group of flavors, fragrances, colors and vitamins.
